(19) United States
(12) Patent Application Publication
(10) Pub. No.: US 2013/0144170 A1
(43) Pub. Date: Jun. 6, 2013

(54) METHOD FOR GENERATING ULTRASONIC IMAGE USING CONCAVE ARRAY

(71) Applicants: Jin Ho Chang, Seoul (KR); Tai-Kyong Song, Seoul (KR)

(72) Inventors: Jin Ho Chang, Seoul (KR); Tai-Kyong Song, Seoul (KR)

(21) Appl. No.: 13/808,427

(22) PCT Filed: Jul. 7, 2011
(86) PCT No.: PCT/KR2011/004951

§ 371 Date: Feb. 20, 2013

(30) Foreign Application Priority Data
Jul. 7, 2010 (KR) 10-2010-0065325

(51) Int. Cl. A61B 8/10

(52) U.S. Cl. CPC A61B 8/10
USPC 600/447

(57) ABSTRACT

Provided is a method of producing an ultrasound image using a concave array, which includes locating a concave array having at least one ultrasound transducer on a cornea, generating an ultrasound image by using an ultrasound transmitted or received through the concave array, and displaying the generated ultrasound image, wherein a focusing point of the concave array is on a vitreous humor, thereby minimizing ultrasound refraction, minimizing a loss of ultrasound energy, and resultantly makes a posterior segment into an ultrasound image.

### TECHNICAL FIELD

The following disclosure relates to a method of producing an ultrasound image using a concave array, and in particular, to a method of producing an ultrasound image using a concave array, which may minimize ultrasound refraction, minimize a loss of ultrasound energy and resultantly make a posterior segment into an ultrasound image by matching a concave surface pattern of the concave array with a pattern of the cornea.

### BACKGROUND

A high frequency ultrasound is an ultrasound (over 20-100 MHz) with operation frequency over 10 times of operation frequency of an existing ultrasound, namely 2-10 MHz.

Ophthalmic image is one of fields where high frequency (HF) ultrasound imaging is applied. A HF ultrasound in the range of 35 MHz to 100 MHz is used for diagnosing an anterior segment including a ciliary body, a zonular fiber frequently appearing at the tumor and the cyst, and an anterior chamber formed by a bond angle between the iris and the cornea, which is important for the study of glaucoma.

The ultrasound in the range of 7 MHz to 20 MHz is used for imaging a posterior segment which is clinically important to diagnose the pathology of a posterior pole such as macular degeneration, detached retina, and retina vein occlusion.

Existing ultrasound has a spatial resolution of several millimeters, but the spatial resolution may be enhanced to several ten micrometers or less if a high frequency ultrasound is used.

At present, an image is obtained while mechanically moving a high frequency single-element converter and used for diagnosing glaucoma, macular degeneration, and retinal detachment of an eye. However, if the single-element converter is used, an ultrasound functional image such as a 2D color flow image is not obtained, which makes it impossible to diagnose a disease such as retina vein occlusion that is the second-largest factor of blindness.

Therefore, even though high operation frequency is necessary to obtain high spatial resolution, since the ultrasound is attenuated in proportion to the increase of frequency, it is difficult to make a posterior segment into an ultrasound image by using high frequency.

In an ophthalmic ultrasound image, another important issue is a loss of ultrasound energy caused by refraction of an anterior segment of the eye. It is because the ultrasound transmitted to the eyeball is greatly refracted at the anterior segment due to a circular eyeball structure. Therefore, conventional ultrasound transducer arrays in a linear or block form are inefficient to transmit an ultrasound to a posterior segment of the eyeball or receive an ultrasound emitted from the posterior segment.

In other words, when generating a retina image by using a linear array converter or a convex array converter, high transmission ultrasound refraction occurs at a cornea and a lens due to the circular shape of the eye, which makes it impossible to obtain a high resolution image at a desired region.

### SUMMARY

An embodiment of the present disclosure is directed to providing a method of producing an ultrasound image using a concave array, which may minimize ultrasound refraction, minimize a loss of ultrasound energy and resultantly make a posterior segment into an ultrasound image by matching a concave surface pattern of the concave array with a pattern of the cornea so that an ultrasound is incident to the cornea of the eye perpendicularly from the concave array.

In one general aspect, a method of producing an ultrasound image using a concave array includes: locating a concave array having at least one ultrasound transducer on a cornea; generating an ultrasound image by using an ultrasound transmitted or received through the concave array; and displaying the generated ultrasound image, wherein a focusing point of the concave array is on a vitreous humor.

According to an embodiment of the present disclosure, in a case where the concave array is located on an eyelid, the focusing point of the concave array may be on a vitreous humor of an eyeball.

In addition, the ultrasound image may be an image of a posterior segment of an eyeball or retina.

According to another embodiment of the present disclosure, the generating of an ultrasound image may generate a scanning line by means of synthetic aperture and generate an ultrasound image by using the generated scanning line.

In addition, the generating of an ultrasound image may generate a scanning line by means of frequency compound imaging or harmonic imaging and generate an ultrasound image by using the generated scanning line.

In addition, the generating of an ultrasound image may generate a scanning line by means of coded excitation, which enhances a signal-to-noise ratio (SNR) by transmitting/receiving and then compressing coded pulses, and generate an ultrasound image by using the generated scanning line.

According to another embodiment of the present disclosure, the focusing point of the concave array may vary by changing a curvature of the concave array. The focusing point of the concave array may be closer to the concave array as the concave array has a greater curvature.

In addition, a curvature of the concave array may be identical to a curvature of the cornea.

In addition, the ultrasound transmitted or received through the concave array may perpendicularly pass through the cornea.

According to the present disclosure, it is possible to minimize ultrasound refraction, minimize a loss of ultrasound energy and resultantly make a posterior segment into an ultrasound image by matching a concave surface pattern of the concave array with a pattern of the cornea so that an ultrasound is incident to the cornea of the eye perpendicularly from the concave array. In addition, according to the present disclosure, by designing an ultrasound converter in a concave surface pattern, it is possible to ensure a desired image width and also obtain a 2D color flow image, which is a functional image, as well as a high resolution anatomical image

### BRIEF DESCRIPTION OF THE DRAWINGS

The above and other objects, features and advantages of the present disclosure will become apparent from the following description of certain exemplary embodiments given in conjunction with the accompanying drawings, in which:

FIG. 1 is a diagram showing a state where a concave array is located at the eyeball;

FIG. 2 is a diagram for calculating a time delay when a beam is focused while transmitting or receiving an ultrasound; and

FIG. 3 is a flowchart for illustrating a method of producing an ultrasound image using a concave array according to an embodiment of the present disclosure.

### DETAILED DESCRIPTION OF EMBODIMENTS

A method of producing an ultrasound image using a concave array includes: locating a concave array having at least one ultrasound transducer on a cornea; generating an ultrasound image by using an ultrasound transmitted or received through the concave array; and displaying the generated ultrasound image, wherein a focusing point of the concave array is on a vitreous humor.

Hereinafter, the present disclosure will be described in more detail based on embodiments of the present disclosure. However, the embodiments of the present disclosure are for better understanding of the present disclosure, and it will be obvious to those having ordinary skill in the art that the scope of the present disclosure is not limited to the embodiments. Moreover, if it is judged that detailed explanation about known functions or configurations in relation to the present disclosure or other matters may make the substance of the present disclosure unnecessarily vague, such detailed explanation will be omitted.

In an ophthalmic ultrasound image, in order to obtain high spatial resolution particularly for a posterior segment image (a retina image of the eye), a high operation frequency is required. However, since an ultrasound is attenuated in proportion to the increase of frequency, it is difficult to make the posterior segment into an ultrasound image. In addition, since the ultrasound emitting from an array causes refraction when being incident to the cornea of the eye, it is difficult to obtain an ultrasound image of the posterior segment. In order to solve such problems, a method of producing an ultrasound image using a concave array according to an embodiment of the present disclosure will be described in detail.

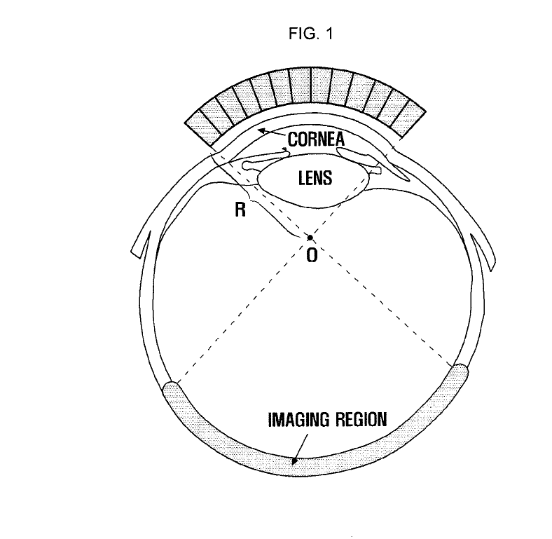

FIG. 1 is a diagram showing a state where a concave array is located at the eyeball.

Referring to FIG. 1, it may be understood that a curvature center O of a concave array is located within the eyeball, and the eyeball is filled with a vitreous humor. In addition, the concave array is composed of at least one ultrasound transducer.

Since the concave array is generally located on the eyelid, it is preferred that the curvature center O is located on the vitreous humor in consideration of the thickness of the eyelid.

If the center of the concave array curvature is located within the eyeball filled with a vitreous humor generally composed of water, the problem caused by reverberation is solved.

When interpreting a beam profile of a concave array transducer, the concave array may be regarded as a linear array having a fixed focusing point. This is because a physical pattern of the concave array transducer allows a transmitted ultrasound to be focused on the curvature center.

Therefore, the beam widths of the concave array in the lateral direction and the axial direction are similar to the beam width of a linear array having a sub-aperture of the same size. However, the concave array and the linear array are different from each other in view of their scan lines.

In other words, while a plane formed by the linear array is a rectangular plane, an image plane formed by the concave array has a fan shape.

Therefore, a scanning transformation process for displaying the received scan lines on a monitor is required. This scanning transformation process may be implemented in a way similar to scanning transformation used in a sector scanning system when a display region is located over the curvature center.

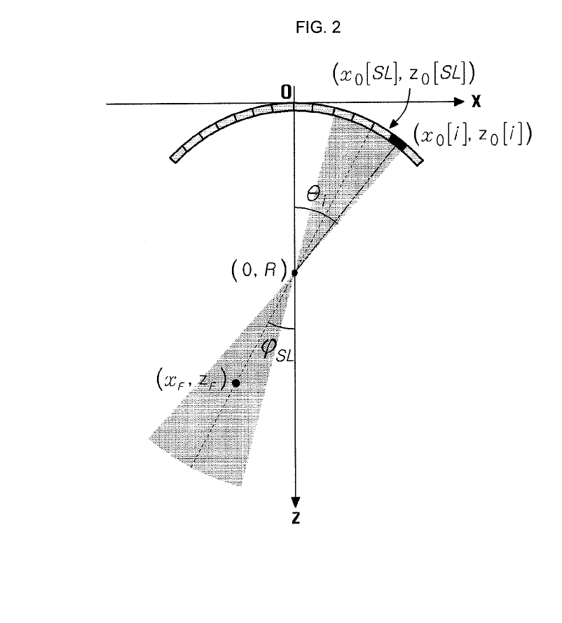

FIG. 2 is a diagram for calculating a time delay when a beam is focused while transmitting or receiving an ultrasound.

Referring to FIG. 2, the sub-aperture has four elements, and the dotted line represents a scan line where a focusing point is located. The scan line is inclined from (x₀[SL], z₀[SL]) as much as φ_{SL} with respect to the z axis. An i^{th} array element is distinguished by the block color, is located at (x₀[i], z₀[i]), and is inclined as much as θᵢ with respect to the z axis.

When focusing a transmitted or received ultrasound beam, the concave surface pattern is used for calculating a delay time of each element of the sub-aperture.

The delay time of each element when focusing a fixed transmitted beam may be calculated according to Equation 1 below.

$$Δ {t}_{TX} = \frac{\begin{matrix}\sqrt{{( {x}_{F} - {x}_{0} [ i ] )}^{2} + {( {z}_{F} - {z}_{0} [ i ] )}^{2}} - \\ \sqrt{{( {x}_{F} - {x}_{0} [ SL ] )}^{2} + {( {z}_{F} - {z}_{0} [ SL ] )}^{2}}\end{matrix}}{c} \qquad Equation 1$$

Here, c represents an ultrasound speed, (x_{F}, z_{F}) represents a location of a focus depth, and (x₀[i], z₀[i]) represents a location of an i^{th} element of the array.

In addition, (x₀[SL], z₀[SL]) represents a start location of a scan line inclined as much as an angle φ_{SL} with respect to the z axis with the curvature center at (0, R).

Each scan line is inclined as much as the angle φ_{SL} and a location of each element may be expressed by a curvature radius R and an angle θᵢ. Therefore, Equation 1 may be simplified into Equation 2 below after rotating the scan line as much as φ_{SL} with respect to the curvature center.

$$Δ {t}_{TX} = \frac{{z}_{F}}{c} - \frac{\sqrt{{{x}_{0}^{′} [ i ]}^{2} + {( {z}_{F} - {z}_{0}^{′} [ i ] )}^{2}}}{c} \\ here {x}_{0}^{′} [ i ] = R \cdot sin ( {θ}_{i} - {ϕ}_{LS} ) , \\ {z}_{0}^{′} [ i ] = R \cdot [ 1 - cos ( {θ}_{i} - {ϕ}_{LS} ) ] . \qquad Equation 2$$

When forming a dynamic received beam, the delay time may be calculated by handling z_{F} as a variable corresponding to a pixel point.

Hereinafter, since it is important to determine pitch, height and size of elements in order to obtain the best spatial resolution in a region of interest, a method of determining a pitch will be described.

A pitch of elements is selected so that a space between two adjacent scan lines is so sufficiently narrow to express a resolution cell of a system.

Since an image generated by the concave array has a fan shape, a criterion of selection used for designing a convex array may be used for designing a concave array by just a little modification.

A pitch P of the convex array satisfies Equation 3 below.

$$P \leq 0.61 \frac{R \cdot {f}_{\#} \cdot λ}{z} = \sqrt{0.61 R \cdot \frac{λ}{N}} \qquad Equation 3$$

Here, f_{#} represents an F-number, A represents frequency, and z represents an image depth. If a single sub-aperture has N number of elements, f_{#} may be expressed as z/N·P.

Since the concave array provides a fan-shaped image plane at the rear of the curvature center, the image depth of Equation 3 should be modified from z to z-R. Therefore, in the concave array, Equation 3 may be modified into Equation 4 below.

$$P \leq \sqrt{0.61 \frac{z \cdot R}{z - R} \cdot \frac{λ}{N}} \qquad Equation 4$$

In the process of modifying Equation 3 into Equation 4, even though f_{#} has a z variable, f_{#} will not be influenced.

Referring to Equations 3 and 4, it may be understood that the concave array has a greater pitch than the convex array.

The pitch between elements is particularly much greater in a high frequency application device where a small array element with low sensitivity is used, which is an advantage of the concave array in comparison to the convex array.

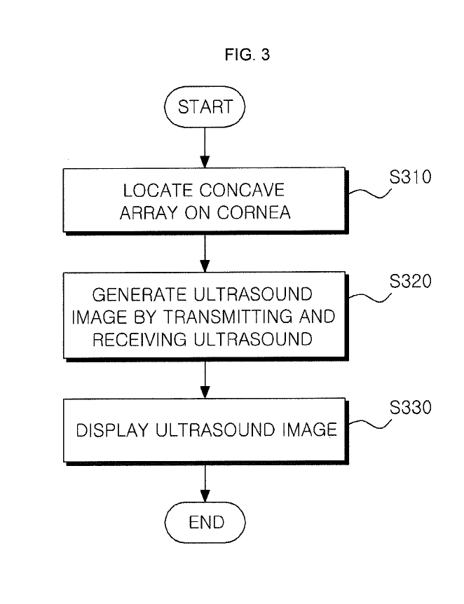

FIG. 3 is a flowchart for illustrating a method of producing an ultrasound image using a concave array according to an embodiment of the present disclosure.

In Operation S310, a concave array is located on the cornea. Generally, the concave array is located on the eyelid, and the concave array preferably has the same curvature as the cornea.

In addition, since the starting point of the curvature of the concave array is located on the vitreous humor, all transmitted ultrasounds are physically focused on a single point. This feature allows minimizing reverberation and ensuring a great image width.

In Operation S320, an ultrasound is transmitted and received through the concave array to generate an ultrasound image.

A ring-type concentric dual-element transducer may generate an ultrasound image, particularly a posterior segment ultrasound image, by means of 20 MHz/40 MHz harmonic imaging and frequency compound imaging. In addition, a rotational scanning method using a 40 MHz angled needle transducer or a synthetic aperture technique may be applied.

The frequency compound imaging is a real-time imaging technique for composing images observed at various angles into a single image. The harmonic imaging is a method using a harmonic frequency generated when an ultrasound passes through a tissue.

Doppler spectrum and color flow imaging are as important as B-mode imaging when generating a posterior segment image.

For the Doppler spectrum or the color flow imaging, as a general device used for ophthalmic imaging equipment using an ultrasound, an electronically translating HF array is required rather than a mechanically translating single element or a ring-type array.

In addition, it is possible to generate a scanning line by means of coded excitation, which transmits or receives coded pulses to/from a target and then compresses the coded pulses to enhance a signal-to-noise ratio (SNR), and then generate an ultrasound image by using the generated scanning line. At this time, the coded pulses may use a chirp sequence or a barker sequence, without being limited thereto.

In Operation S330, the generated ultrasound image is displayed.

According to another embodiment of the present disclosure, the present disclosure may be applied to a case where the surface of a target has a concave surface pattern, regardless of a portion where the curvature center O of the concave array is located, without being limited to the case where the curvature center O of the concave array is located in a human body region such as the vitreous humor, which is generally composed of water.

At this time, the curvature center O of the concave array is preferably identical to the curvature center O of the surface of the target, or within a certain error range.

If the curvature center O of the concave array is a human body region such as the vitreous humor, which is generally composed of water, the problem caused by reverberation is solved as described above. However, in case of obtaining an ultrasound image of the heart, since the curvature center O of the concave array is located at the rib, reverberation is serious and it is not easy to obtain a desired image.

According to another embodiment of the present disclosure, the reverberation problem may be solved by generating an ultrasound in a human body without transmitting an ultrasound. For this, photoacoustic imaging (PAI) is used.

The photoacoustic imaging is a technique of obtaining an image by transmitting laser pulses into a human body to generate an ultrasound in the human body and then receiving the generated ultrasound through an ultrasound converter. Since the photoacoustic imaging does not need to transmit an ultrasound, if the concave array converter according to an embodiment of the present disclosure is used for receiving an ultrasound in the photoacoustic imaging, the ultrasound generated in the human body may be efficiently received while minimizing a loss caused by refraction of the received energy.

The method of producing an ultrasound image using a concave array according to an embodiment of the present disclosure, which uses the photoacoustic imaging, may be utilized for diagnosing breast cancer, lymph node cancer or the like.

Though the present disclosure has been described based on limited embodiments and drawings as well as specific matters such as detailed components, they are just for better understanding of the present disclosure, and the present disclosure is not limited to the embodiments but various changes and modifications can be made to the present disclosure by those having ordinary skill in the art. Therefore, the scope of the present disclosure should not be limited to the above embodiments but equivalents within the scope of the appended claims should be interpreted as belong to the present disclosure.

What is claimed is:

1. A method of producing an ultrasound image using a concave array, comprising:
locating a concave array having at least one ultrasound transducer on a cornea;
generating an ultrasound image by using an ultrasound transmitted or received through the concave array; and
displaying the generated ultrasound image,
wherein a focusing point of the concave array is on a vitreous humor.

2. The method of producing an ultrasound image using a concave array according to claim 1,
wherein in a case where the concave array is located on an eyelid, the focusing point of the concave array is on a vitreous humor of an eyeball.

3. The method of producing an ultrasound image using a concave array according to claim 1,
wherein the ultrasound image is an image of a posterior segment of an eyeball.

4. The method of producing an ultrasound image using a concave array according to claim 1,
wherein said generating of an ultrasound image generates a scanning line by means of synthetic aperture and generates an ultrasound image by using the generated scanning line.

5. The method of producing an ultrasound image using a concave array according to claim 1,
wherein said generating of an ultrasound image generates a scanning line by means of frequency compound imaging and generates an ultrasound image by using the generated scanning line.

6. The method of producing an ultrasound image using a concave array according to claim 1,
wherein said generating of an ultrasound image generates a scanning line by means of harmonic imaging and generates an ultrasound image by using the generated scanning line.

7. The method of producing an ultrasound image using a concave array according to claim 1,
wherein said generating of an ultrasound image generates a scanning line by means of coded excitation, which enhances a signal-to-noise ratio (SNR) by transmitting/receiving and then compressing coded pulses, and generates an ultrasound image by using the generated scanning line.

8. The method of producing an ultrasound image using a concave array according to claim 1,
wherein the focusing point of the concave array varies by changing a curvature of the concave array.

9. The method of producing an ultrasound image using a concave array according to claim 1,
wherein a curvature of the concave array is identical to a curvature of the cornea.

10. The method of producing an ultrasound image using a concave array according to claim 1,
wherein the ultrasound transmitted or received through the concave array perpendicularly passes through the cornea.

11. The method of producing an ultrasound image using a concave array according to claim 1,
wherein the focusing point of the concave array is closer to the concave array as the concave array has a greater curvature.

12. A method of producing an ultrasound image using a concave array, comprising:
locating a concave array having at least one ultrasound transducer on a target;
when an ultrasound is generated by means of photoacoustic imaging (PAI), generating an ultrasound image by using an ultrasound transmitted or received through the concave array; and
displaying the generated ultrasound image,
wherein the target and the concave array have the same curvature.
